(19) United States
(12) Patent Application Publication
(10) Pub. No.: US 2014/0314702 A1
(43) Pub. Date: Oct. 23, 2014

(54) COMPOSITION

(71) Applicants: Qunhua Cao, Shanghai (CN); Sungmin Cho, Gyeonggi-Do (KR); Amit Jayaswal, Shanghai (CN); Jihye Lee, Gyeonggi-Do (KR); Jianming Liang, Taizhou City (CN); Liansong Xie, Shanghai (CN); Qing Yang, Shanghai (CN); Shuhong Yuan, Shanghai (CN)

(72) Inventors: Qunhua Cao, Shanghai (CN); Sungmin Cho, Gyeonggi-Do (KR); Amit Jayaswal, Shanghai (CN); Jihye Lee, Gyeonggi-Do (KR); Jianming Liang, Taizhou City (CN); Liansong Xie, Shanghai (CN); Qing Yang, Shanghai (CN); Shuhong Yuan, Shanghai (CN)

(21) Appl. No.: 14/239,659

(22) PCT Filed: Aug. 13, 2012
(86) PCT No.: PCT/EP2012/065783

§ 371 Date: Jun. 12, 2014

(30) Foreign Application Priority Data
Aug. 25, 2011 (CN) PCT/CN2011/078901
Jul. 3, 2012 (CN) PCT/CN2012/078121

(51) Int. Cl. A61K 8/58; A61Q 5/12

(52) U.S. Cl. CPC A61K 8/58; A61Q 5/12
USPC 424/70.11; 424/70.1; 424/70.21; 424/70.27; 424/70.28; 424/70.31

(57) ABSTRACT

Hair conditioning composition comprising: —from 0.1 to 10% wt. silicone —from 0.1 to 5% wt. metal pyrithione blended with non-anionic surfactant.

The present invention relates to a stable hair conditioning composition comprising metal pyrithione.

Despite the prior art there remains a need for improved hair conditioning compositions.

Accordingly, the present invention provides, in a first aspect, a hair conditioning composition comprising:
from 0.1 to 10% wt. silicone; and
from 0.1 to 5% wt. metal pyrithione blended with non-anionic surfactant.

The composition of the invention does not foul process machinery and provides an anti-dandruff benefit.

In a further aspect, the present invention provides a process for manufacturing a hair conditioning composition comprising the steps of:
i) forming a blend comprising metal pyrithione and non-anionic surfactant; and
ii) combining the blend with a base composition comprising silicone.

The process of the invention can suitably be used to manufacture the hair conditioning composition of the first aspect of the invention.

In a still further aspect, the present invention provides a blend comprising:
(a) 10 to 89% by weight of the blend of metal pyrithione; and
(b) 0.1 to 10% by weight of the blend of non-anionic surfactant.

The blend can suitably be used in the process and/or the composition of the invention.

Except in the examples, or where otherwise explicitly indicated, all numbers in this description indicating amounts of material or conditions of reaction, physical properties of materials and/or use may optionally be understood as modified by the word “about”.

All amounts are by weight of the final hair conditioning composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a process of the invention) mutatis mutandis.

The present invention employs non-anionic surfactant as part of a blend with metal pyrithione. By “non-anionic surfactant” is meant surfactant selected from cationic surfactant, nonionic surfactant, amphoteric surfactant, or a mixture thereof.

Preferably the non-anionic surfactant is, or at least comprises, cationic surfactant. Preferred cationic surfactants are quaternary ammonium salts. More preferably, the cationic surfactant has the formula N⁺R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. Alkyl groups may be straight chain or branched and, for alkyl groups having 3 or more carbon atoms, cyclic. The alkyl groups may be saturated or may contain one or more carbon-carbon double bonds (eg, oleyl). Preferably, the alkyl groups comprise one or more ester (—OCO— or —COO—) and/or ether (—O—) linkages within the alkyl chain. More preferably, the alkyl groups comprise one or more ether linkages within the alkyl chain. The most preferred cationic surfactant is benzethonium chloride.

Preferably the non-anionic surfactant is, or at least comprises, non-ionic surfactant. Thus in a preferred embodiment the invention provides a hair conditioning composition comprising:
from 0.1 to 10% wt. silicone; and
from 0.1 to 5% wt. metal pyrithione blended with non-ionic surfactant.

Preferred non-ionic surfactants are those comprising ether and/or ester. The ether may, for example, comprise condensation product of aliphatic (C₈-C₁₈) primary or secondary linear or branched chain alcohol or aromatic alcohol with alkylene oxide, usually ethylene oxide and generally having from 2 to 30 ethylene oxide groups. Alkyl ethoxylates are particularly preferred. More preferred are alkyl ethoxylates having the formula R—(OCH₂CH₂)ₙOH, where R is H or an alkyl chain, and n is 6 to 20. Aromatic alcohol preferably comprises phenol or naphtol.

Suitable ester includes, for example, glycerol alkyl ester, polyoxyethylene glycol sorbitan alkyl ester, sorbitan alkyl ester. Preferably, the alkyl group has 4 to 30 carbon atoms. The preferred esters are ethoxylated novolak esters, polyoxyethylene glycol sorbitan alkyl ester, more preferably polysorbate. Even more preferred is that the nonionic surfactant comprises ethoxylated novolak ester, polyoxyethylene β-naphtol ether, poly(oxy-1,2-ethanediyl), polysorbate 80, or a mixture thereof.

In a most preferred embodiment, the non-ionic surfactant is, or at least comprises, ethoxylated novolak ester.

Preferably, the ethoxylated novolak ester is a nonylphenol ethoxylated ester. An example of a suitable novolak ester is Dispersogen 2774 ex. Clariant.

Preferably the non-anionic surfactant is, or at least comprises, amphoteric surfactant. Preferably the amphoteric surfactant comprises, for example, alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms. Particularly preferred amphoteric surfactant comprises alkyl amphoacetates, wherein the alkyl group has 8 to 19 carbon atoms. The most preferred amphoteric surfactant is disodium cocoamphoacetate.

Preferably, the non-anionic surfactant comprises from 0.1 to 10% wt. of the blend, more preferably from 0.5 to 2.5% wt.

The blend is preferably substantially free from anionic surfactant. More preferably, less than 10% of surfactant in the blend by weight of the surfactant in blend is anionic surfactant. Even more preferably, less than less than 3%, and more preferably still less than 1% of surfactant in the blend by weight of the surfactant in blend is anionic surfactant. Most preferably, from 0 to 0.1% of surfactant in the blend by weight of the surfactant in blend is anionic surfactant.

The metal pyrithione for use in the present invention typically comprises zinc pyrithione, copper pyrithione, silver pyrithione, zirconium pyrithione, or a mixture thereof. Most preferably, the metal pyrithione is zinc pyrithione.

Preferably, the metal pyrithione comprises from 10 to 89% wt. of the blend, more preferably from 30 to 70% wt. and most preferably from 40 to 60% wt.

Preferably, the blend also comprises water at from 10 to 89% wt. of the blend, more preferably from 30 to 70% wt. and most preferably from 40 to 60% wt.

The blend may also comprise other substances. In a preferred embodiment, the blend further comprises polymer, especially polymer which comprises at least 90% of nonionic polymer by weight of the polymer. The polymer is preferably different from the surfactant. More preferably, the polymer is a rheological modifier, even more preferably, the polymer is a thickener.

Preferably from 95% to 100% by weight of polymer is nonionic polymer. Most preferably the polymer is nonionic polymer.

Additionally or alternatively, the polymer is substantially free from cationic polymer. More preferably, less than 5% by weight of polymer is cationic polymer. Even more preferably, less than 1% by weight of polymer is cationic polymer. Most preferably, less than 0.1% by weight of polymer is cationic polymer.

In fact we have also found that the presence of cationic polymer in the blend may increase silicone fouling, thus in a preferred embodiment, whether or not the blend comprises non-ionic polymer, it is preferred that the blend comprises less than 0.1% cationic polymer by weight of the blend, more preferably less than 0.01%, and most preferably from 0 to 0.001%.

The weight-average molecular weight of the polymer for use in the premix of the present invention is preferably in the range from 1,000 to 2,000,000, more preferably from 8,000 to 1,000,000, even more preferably from 20,000 to 500,000.

Preferably, the nonionic polymer is nonionic hydrophobically-modified polymer. “Hydrophobically-modified” as used herein refers to modification to improve the hydrophobicity of the polymer, for example, by attaching alkyl and/or aralkyl group.

The nonionic polymer suitable for use in the blend of the present invention includes, for example, hydrophobically modified nonionic hydroxyalkyl cellulose polymer, hydrophobically modified nonionic polyol, nonionic hydrophobically-modified urethane polymer, or a mixture thereof. More preferably, the nonionic polymer is or at least comprises nonionic hydrophobically-modified urethane polymer. The nonionic polymer also preferably comprises polglycol ester. The most preferred nonionic polymer is nonionic hydrophobically-modified ethoxylated urethane.

The preferred nonionic hydrophobically-modified ethoxylated urethane has the following general structure:

R₂O-Eₙ-[CO—NH—R₁—NH—CO—O-Eₙ-CO—NH—R₁—NH—CO]ₓ-Eₙ-OR₂,

wherein Eₙ is polyethylene glycol with structure (CH₂CH₂O)ₙ, n is preferably in the range from 20 to 2000, more preferably from 50 to 500; R₁ and R₂ are independently selected from straight or branched chain alkyl, alkenyl, or aromatic group. Optionally R₁ and/or R₂ contain functional group such as for example COOH, NH₂ and/or OH.

The most preferred nonionic hydrophobically-modified ethoxylated urethane has ICNI name of PEG-150/steryl alcohol/SMDI copolymer.

Suitable nonionic polymer of the present invention includes, for example, Aculyn™ 46, and Aculyn™ 44 from Dow Chemical.

Typically, the nonionic polymer is present in the blend in amount from 0.01 to 10% by weight of the blend, more preferably from 0.05 to 6% by weight of the blend, even more preferably from 0.2 to 3% by weight of the blend, and most preferably from 0.4 to 2% by weight of the blend.

In order to maximise stability of the blend, the weight ratio of non-anionic surfactant to polymer in the blend is preferably in the range from 1:20 to 20:1, more preferably from 1:10 to 10:1, and even more preferably from 1:5 to 5:1.

Preferably, the blend is present at from 0.1 to 5% wt. of the composition, more preferably from 0.15 to 2.5% wt. of the composition.

The composition of the invention comprises silicone. Preferably, the composition of the invention contains emulsified droplets of a silicone conditioning agent, for enhanced conditioning performance.

The silicone is preferably a non-volatile silicone, wherein non-volatile silicone means that the silicone has vapor pressure less than 0.1 mm Hg (13.3 Pa), preferably less than 0.05 mm Hg, more preferably less than 0.01 mm Hg at 25° C. and atmospheric pressure.

Suitable silicones include polydiorganosiloxanes, in particular polydimethylsiloxanes which have the CTFA designation dimethicone. Also suitable for use compositions of the invention are polydimethyl siloxanes having hydroxyl end groups, which have the CTFA designation dimethiconol. Also suitable for use in compositions of the invention are silicone gums having a slight degree of cross-linking, as are described for example in WO 9631188 (UNILEVER).

The viscosity of the emulsified silicone itself is typically at least 10,000 cSt at 25° C. The viscosity of the silicone itself is preferably at least 60,000 cSt, most preferably at least 500,000 cSt, ideally at least 1,000,000 cSt. Preferably the viscosity does not exceed 10⁹ cSt for ease of formulation.

Viscosity of silicone can be determined, for example, by the relevant international standard, such as ISO 3104.

Emulsified silicones for use in the compositions of the invention will typically have a size in the composition of less than 30, preferably less than 20, more preferably less than 15 μm. Preferably the average silicone droplet is greater than 0.5 μm, more preferably greater than 1 μm, ideally from 2 to 8 μm.

Silicone particle size may be measured by means of a laser light scattering technique, for example using a 2600D Particle Sizer from Malvern Instruments.

Examples of suitable pre-formed emulsions include Xiameter™ MEM 1785 and microemulsion DC2-1865 available from Dow Corning. These are emulsions microemulsions of dimethiconol. Cross-linked silicone gums are also available in a pre-emulsified form, which is advantageous for ease of formulation.

A further preferred class of silicones for inclusion in the conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”, Specific examples of amino functional silicones suitable for use in the invention are the aminosilicone oils DC2-8220, DC2-8166 and DC2-8566 (all ex Dow Corning).

Suitable quaternary silicone polymers are described in EP-A-0 530 974 (UNILEVER). A preferred quaternary silicone polymer is K3474, ex Goldschmidt.

Also suitable are emulsions of amino functional silicone oils with non ionic and/or cationic surfactant.

Pre-formed emulsions of amino functional silicone are also available from suppliers of silicone oils such as Dow Corning and General Electric. Specific examples include DC939 Cationic Emulsion and the non-ionic emulsions DC2-7224, DC2-8467, DC2-8177 and DC2-8154 (all ex Dow Corning).

The total amount of silicone is preferably from 0.01 wt % to 10% wt of the total composition more preferably from 0.1 wt % to 5 wt %, most preferably 0.5 wt % to 3 wt % is a suitable level.

Preferably, the composition further comprises conditioning surfactants including those selected from cationic surfactants, used singly or in admixture. Preferably, the cationic surfactants have the formula N⁺R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethyleneoxy groups.

Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN KDMP, ex Clariant.

Another example of a class of suitable cationic surfactants for use in the invention, either alone or together with one or more other cationic surfactants, is a combination of (i) and (ii) below:

(i) an amidoamine corresponding to the general formula (I):

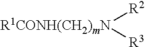

in which R¹ is a hydrocarbyl chain having 10 or more carbon atoms,
R² and R³ are independently selected from hydrocarbyl chains of from 1 to 10 carbon atoms, and m is an integer from 1 to about 10; and
   (ii) an acid.

As used herein, the term hydrocarbyl chain means an alkyl or alkenyl chain.

Preferred amidoamine compounds are those corresponding to formula (I) in which

R¹ is a hydrocarbyl residue having from about 11 to about 24 carbon atoms,
R² and R³ are each independently hydrocarbyl residues, preferably alkyl groups, having from 1 to about 4 carbon atoms, and m is an integer from 1 to about 4.

Preferably, R² and R³ are methyl or ethyl groups.

Preferably, m is 2 or 3, i.e. an ethylene or propylene group.

Preferred amidoamines useful herein include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof.

Particularly preferred amidoamines useful herein are stearamidopropyldimethylamine, stearamidoethyldiethylamine, and mixtures thereof.

Commercially available amidoamines useful herein include: stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

A protonating acid may be present. Acid may be any organic or mineral acid which is capable of protonating the amidoamine in the conditioner composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, lactic acid and mixtures thereof.

The primary role of the acid is to protonate the amidoamine in the hair treatment composition thus forming a tertiary amine salt (TAS) in situ in the hair treatment composition. The TAS in effect is a non-permanent quaternary ammonium or pseudo-quaternary ammonium cationic surfactant.

Suitably, the acid is included in a sufficient amount to protonate more than 95 mole % (293 K) of the amidoamine present.

In conditioners of the invention, the level of cationic surfactant will generally range from 0.01% to 10%, more preferably 0.05% to 7.5%, most preferably 0.1% to 5% by weight of the composition.

Compositions according to the present invention preferably also comprise a dispersed, non-volatile, water-insoluble oily conditioning agent which is not a silicone.

By “insoluble” is meant that the material is not soluble in water (distilled or equivalent) at a concentration of 0.1% (w/w), at 25° C.

Suitable materials include oily or fatty materials selected from hydrocarbon oils, fatty esters and mixtures thereof. Straight chain hydrocarbon oils will preferably contain from about 12 to about 30 carbon atoms. Also suitable are polymeric hydrocarbons of alkenyl monomers, such as C₂-C₆ alkenyl monomers.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used.

Suitable fatty esters are characterised by having at least 10 carbon atoms, and include esters with hydrocarbyl chains derived from fatty acids or alcohols, Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are mono-, di- and triglycerides, more specifically the mono-, di-, and tri-esters of glycerol and long chain carboxylic acids such as C₁-C₂₂ carboxylic acids. Preferred materials include cocoa butter, palm stearin, sunflower oil, soyabean oil and coconut oil.

The oily or fatty material is suitably present at a level of from 0.05 wt % to 10 wt %, preferably from 0.2 wt % to 5 wt %, more preferably from about 0.5 wt % to 3 wt %.

Conditioners of the invention will preferably also incorporate a fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol in conditioners of the invention will generally range from 0.01 to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, more preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

We have found that the problem of silicone fouling can be reduced by the present invention even where the silicones comprise very little, or no, cyclopentasiloxane. Therefore in one embodiment the hair conditioning composition preferably comprises from 0 to 0.5% wt. cyclopentasiloxane, more preferably from 0 to 0.1% wt, more preferably still from 0 to 0.01% wt. Most preferably the composition is free from cyclopentasiloxane.

The hair conditioning composition may be manufactured by any suitable process but it is particularly preferred that it is manufactured using a process comprising the steps of:
i) forming a blend comprising metal pyrithione and non-anionic surfactant; and
ii) combining the blend with a base composition comprising silicone.

Preferably, the step (i) comprises the treatment of milling; more preferably comprises the treatment of milling and filtering. Preferably, the treatment of milling is conducted for from 1 to 100 hours.

Preferably, the base composition comprises, in addition to silicone, an ingredient selected from water, fatty alcohol, cationic surfactant, or a mixture thereof.

Preferably the blend and the base composition are combined in step (ii) in a weight ratio of blend:base composition in the range from 1:10000 to 100:1, more preferably from 1:2000 to 5:1, even more preferably from 1:500 to 1:2, most preferably from 1:100 to 1:10.

The invention will now be described with reference to the following non-limiting examples.

### EXAMPLE 1

The following formulation given in Table 1 is made by standard processes.

TABLE 1

| Ingredient | % wt. |
|---|---|
| Behenyltrimmonium chloride | 0.875 |
| Stearamidopropyl dimethylamine | 1.25 |
| Cetearyl alcohol | 5.00 |
| Aminosilicone | 3.00 |
| Zinc pyrithione and Dispersogen 2774 blend | 0.50 |
| Zinc sulphate | 0.01 |
| Perfume | 0.75 |
| Thickener | 0.30 |
| Lactic acid | 0.38 |
| Preservatives | 0.22 |
| Water | To 100 |

### EXAMPLE 2

A suitable blend for use in the present invention was manufactured as follows:

Zinc pyrithione 700 g, deionized water 700 g and Dispersogen 2774 (ex. Clariant GmbH) 18.2 g were added into a ball mill, milled for 12 hours, then pumped out as a suspension, filtered with 240 mesh filter cloth to obtain blended zinc pyrithione in aqueous dispersion.

### EXAMPLE 3

An experiment comparing silicone fouling of machinery used during manufacture shows that the fouling of machinery by silicone deposits is significant when using standard ZPTO as used in shampoo compositions and significantly absent when using the ZPTO non-ionic surfactant blend as described in Example 2.

### EXAMPLE 4

This example demonstrates the preparation of blends of the present invention.

TABLE 2

| | Blend | | | | | | | |
|---|---|---|---|---|---|---|---|---|
| Ingredient (wt %) | 1 | 2 | 3 | 4 | 5 | 6 | 7 | 8 |
| Zinc pryithione | 27.2 | 35.2 | 40 | 50 | 26.4 | 26.7 | 26.8 | 26.4 |
| Benzethonium | 0.5 | 1 | 1.5 | 1.5 | — | — | | |
| chloride | | | | | | | | |
| Polysorbate 80 | — | — | — | — | 3 | — | — | — |
| Polyoxyethylene β- | — | — | — | — | — | 0.5 | — | — |
| naphtol ether | | | | | | | | |
| poly(oxy-1,2- | — | — | — | — | — | — | 2 | — |
| ethanediyl) | | | | | | | | |
| Disodium | — | — | — | — | — | — | — | 0.9 |
| cocoamphoacetate | | | | | | | | |
| PEG-150/steryl | 1.5 | 1.5 | 0.5 | 1.5 | 1.5 | 1.64 | 1.5 | 1.64 |
| alcohol/SMDI | | | | | | | | |
| copolymer* | | | | | | | | |
| Water | to 100 | to 100 | to 100 | to 100 | to 100 | to 100 | to 100 | to 100 |

*Aculyn 46 from Dow Chemical

The amount of zinc pyrithione, surfactant, PEG-150 steryl alcohol/SMDI copolymer, and deionized water were weighed according to the Table 2. These ingredients were added into a ball mill, milled 12 hours, then the suspension pumped out, filtered with 240 mesh filter cloth to obtain blends of the present invention.

### EXAMPLE 5

This example demonstrates the preparation of control sample.

Control samples were prepared according to the similar procedure as described above in Example 4. The ingredient and concentration followed Table 3. These control samples contain anionic surfactant (sodium poly(naphthalene-formaldehyde) sulfonate.

TABLE 3

| | Control | | | |
|---|---|---|---|---|
| Ingredient (wt %) | A | B | C | D |
| Zinc pryithione | 30 | 30 | 30 | 30 |
| Sodium Poly(naphthalene- | 0.8 | 0.8 | 0.8 | 0.8 |
| formaldehyde) sulfonate | | | | |
| Cocamidopropyl betaine | 0.9 | — | — | — |
| Polyoxyethylene β-naphtol | — | 3.0 | 6.0 | — |
| ether | | | | |
| Disodium cocoamphoacetate | — | — | — | 3.0 |
| Water | to 100 | to 100 | to 100 | to 100 |

### EXAMPLE 6

This example demonstrated silicone fouling was significantly decreased by the blends of the present invention.

The blends of Example 4 and control samples of Example 5 were formulated to hair treatment compositions having an identical composition except for blend components. Then, these compositions were used to test the silicone fouling. The test of silicone fouling was conducted by mimicking the cleaning step of equipment in factory and following the steps in sequence of:
1. Pouring 800 g of testing composition into a stainless beaker (1000 ml);
2. Stirring at 160 rpm for 20 min by a flat paddle (Heidolph Instrument, Germany), then pouring out 700 g of the testing liquid through a stainless sieve with size of 710 μm and replacing the same weight of hot water (80 to 90° C.) into the stainless beaker; and
3. Repeating step 2 for three times.
4. Drying the beaker, paddle, and sieve at 45° C. for 3 hours.

The value of silicone fouling is calculated as the ratio of the weight increase of the dried beaker, paddle, and stainless sieve to the amount of silicone in testing composition by weight.

TABLE 4

| Sample | | Silicone fouling (%) |
|---|---|---|
| Hair treatment composition | 1 | 0.81 |
| with blend | 2 | 0.44 |
| | 3 | 0.32 |
| | 4 | 3.57 |
| | 5 | 1.14 |
| | 6 | 0.26 |
| | 7 | 0.73 |
| | 8 | 0.76 |
| Hair treatment composition | A | Not measured* |
| with control | B | Not measured* |
| | C | 6.67 |
| | D | 16.25 |

*“Not measured” as used herein means even a stable formulation can not be formed owning to visible precipitation of particles.

As can be seen from Table 4, the smallest value of silicone fouling caused by hair treatment composition with control blend is 6.67. In contrast, the biggest value of silicone fouling by caused by hair treatment composition with blend of the invention when used in an identical composition is 3.57. It is demonstrated that the blend of the present invention is unexpectedly more compatible with hair treatment composition. Using the blends of the present invention can dramatically reduce the silicone fouling of hair treatment composition.

### EXAMPLE 7

This example demonstrated the hair/scalp care compositions of the present invention. These compositions were made by standard processes with the formulations given in Table 5.

TABLE 5

| | Composition | | | |
|---|---|---|---|---|
| Ingredient (wt %) | I | II | III | IV |
| Behenyltrimmonium chloride | 0.875 | 0.875 | 0.875 | 0.875 |
| Stearamidopropyl | 1.25 | 1.25 | 1.25 | 1.25 |
| dimethylamine | | | | |
| Cetearyl alcohol | 5.00 | 5.00 | 5.00 | 5.00 |
| Silicone | 3.00 | 3.00 | 3.00 | 3.00 |
| Blend 1 | 0.91 | — | — | — |
| Blend 2 | — | 0.71 | — | — |
| Blend 3 | — | — | 0.63 | — |
| Blend 4 | — | — | | 0.5 |
| Zinc sulphate | 0.1 | 0.1 | 0.1 | 0.1 |
| Sodium chloride | 0.1 | 0.1 | 0.1 | 0.1 |
| Perfume | 0.6 | 0.6 | 0.6 | 0.6 |
| Thickener | 0.20 | 0.20 | 0.20 | 0.20 |
| Lactic acid | 0.38 | 0.38 | 0.38 | 0.38 |
| Preservatives | 0.31 | 0.31 | 0.31 | 0.31 |
| Water | to 100 | to 100 | to 100 | to 100 |

1. Hair conditioning composition comprising:
from 0.1 to 10% wt. silicone; and
from 0.1 to 5% wt. metal pyrithione blended with non-anionic surfactant,
wherein the metal pyrithione comprises from 10 to 89% wt, of the blend;
the non-anionic surfactant comprises from 0.1 to 10% wt, of the blend; and less than 10% of surfactant in the blend by weight of surfactant in the blend is anionic surfactant.

2. Hair conditioning composition according to claim 1, wherein the non-anionic surfactant comprises non-ionic surfactant.

3. Hair conditioning composition according to claim 2, wherein the non-ionic surfactant is selected from an ethoxylated novolak ester, polyoxyethylene β-naphtol ether, poly(oxy-1,2-ethanediyl), polysorbate 80, or a mixture thereof.

4. Hair conditioning composition according to claim 3 wherein the non-anionic surfactant is ethoxylated novolak ester, preferably nonylphenol ethoxylated ester.

5. Hair conditioning composition according to claim 1, wherein the non-anionic surfactant comprises cationic surfactant.

6. Hair conditioning composition according to claim 5, wherein the cationic surfactant is a quaternary ammonium salt, preferably benzethonium chloride.

7. Hair conditioning composition according to claim 1, wherein the non-anionic surfactant comprises amphoteric surfactant, preferably wherein the amphoteric surfactant is disodium cocoamphodiacetate.

8. Hair conditioning composition according to claim 1 comprising a cationic conditioning surfactant.

9. Hair conditioning composition according to claim 1 comprising from 0 to 0.5% wt. cyclopentasiloxane.

10. Hair conditioning composition according to claim 1 comprising aminosilicone.

11. Process for manufacturing a hair conditioning composition comprising the steps of:
i) forming a blend comprising metal pyrithione and non-anionic surfactant; and
ii) combining the blend with a base composition comprising silicone,
wherein the metal pyrithione comprises from 10 to 89% wt, of the blend;
the non-anionic surfactant comprises from 0.1 to 10% wt. of the blend; and less than 10% of surfactant in the blend by weight of surfactant in the blend is anionic surfactant.

12. Process as claimed in claim 11, wherein the blend and the base composition are combined in step (ii) in a weight ratio of blend:base composition in the range of from 1:2000 to 1:2.

13. (canceled)

14. (canceled)

15. Process as claimed in claim 11, wherein the base composition comprises water, fatty alcohol, cationic surfactant, or a mixture thereof.

16. Process as claimed in claim 1 wherein the base composition comprises cationic surfactant.

17. A blend comprising:
(a) 10 to 89% by weight of the blend of metal pyrithione; and
(b) 0.1 to 10% by weight of the blend of non-anionic surfactant;
wherein less than 10% of surfactant in the blend by weight of surfactant in the blend is anionic surfactant.
